(19) United States
(12) Patent Application Publication
(10) Pub. No.: US 2011/0160479 A1
(43) Pub. Date: Jun. 30, 2011

(54) PROCESS FOR THE PRODUCTION OF ESTERS OF LEVULINIC ACID FROM BIOMASSES

(75) Inventors: Daniele Bianchi, Milano (IT); Anna Maria Romano, Novara (IT)

(73) Assignee: POLIMERI EUROPA S.p.A., SAN DONATO MILANESE, Milano (IT)

(21) Appl. No.: 13/000,498

(22) PCT Filed: Jun. 19, 2009
(86) PCT No.: PCT/IB09/06070

§ 371 Date: Mar. 15, 2011

(30) Foreign Application Priority Data
Jun. 26, 2008 (IT) MI2008A001164

(51) Int. Cl. C07C 67/42

(52) U.S. Cl. 560/174

(57) ABSTRACT

A new process is described for the direct synthesis of esters of levulinic acid starting from biomasses containing one or more polysaccharides and from aliphatic alcohols, in the presence of a suitable organic acid catalyst. In particular, the biomasses can be lignin-cellulose biomasses. The esters thus obtained can be used as oxygenated components in the formulation of fuels for motor vehicles, with the aim of reducing the particulate emissions.

The present invention describes a new process for the direct synthesis of esters of levulinic acid starting from biomasses containing one or more polysaccharides and from aliphatic alcohols, in the presence of an acid catalyst. In particular, the biomasses can be lignin-cellulose biomasses. The esters thus obtained can be used as oxygenated components in the formulation of fuels for motor vehicles, with the aim of reducing the particulate emissions.

Generally speaking, a biomass is any substance with an organic, vegetable or animal matrix, which can be used as raw material for energy purposes, for example for the production of biofuels or fuel components. Biomasses can therefore form a renewable energy source as an alternative to traditional fuels of a fossil origin. For this purpose, biomasses containing polysaccharides and especially lignin-cellulose biomasses are particularly useful.

Lignin-cellulose biomasses, which can be used as raw material in the production process of esters of levulinic acid, can be obtained for example from specific crops for energy use or agricultural, forest residues and urban waste. Lignin-cellulose biomasses contain three main components: cellulose, hemicellulose and lignin. The relative quantities vary according to the species and age of the plant.

Cellulose is the greatest constituent of lignin-cellulose biomasses (30-60% by weight) and consists of linear chains of glucose bound through bonds of the 1-4 β-glucoside type.

Hemicellulose forms 10-40% of the lignin-cellulose biomass, it appears as a mixed polymer made up of sugars with six carbon atoms, among which glucose, and sugars with five carbon atoms. Lignin forms 10-30% of the mass.

Sugars with six carbon atoms and in particular the glucose present in the biomasses can be transformed into levulinic acid by acid-catalyzed dehydration to give hydroxymethylfurfural, and subsequent re-arrangement with the formation of levulinic acid and formic acid. The reaction can be carried out, as described in DE 3621517, using an alcohol as solvent for obtaining the corresponding ester of levulinic acid as main product, but the process, which is aimed at the direct transformation of the biomass into esters of levulinic acid, is limited by the low yields. The acid catalysts used in said process, preferably H₂SO₄, in fact, have a low selectivity and catalyze secondary reactions which lead to the degradation of the products with the formation of polymeric carbonaceous residues. In particular, DE 3621517 describes the use of sulfuric acid (1%) at 190-200° C. in ethanol or n-butanol with molar yields into the corresponding esters of levulinic acid (calculated with respect to the glucose) equal to 35 and 39%. The use, as alcohol, of methyl and n-propyl alcohol is illustrated, and p-toluenesulfonic acid, as acid catalyst, but in this case the yield is even lower, equal to 26.7%.

Alternatively, the esters of levulinic acid can be produced by means of two-step processes which envisage an initial hydrolysis of the lignin-cellulose biomass to give levulinic acid, followed by an esterification step. Examples of hydrolysis processes of biomasses to levulinic acid are described in the patents U.S. Pat. No. 560,815 (with a yield to levulinic acid equal to 63%), U.S. Pat. No. 4,897,497 (with a yield to levulinic acid equal to 74%), U.S. Pat. No. 5,892,107 (with a yield to levulinic acid equal to 30%) and U.S. Pat. No. 6,054,611 (with a yield to levulinic acid equal to 60%).

An example of the esterification of levulinic acid with C5-C12 aliphatic alcohols is provided in patent WO 2005/070867, with yields equal to 85%. Alternatively, the levulinic acid can be esterified using olefins (such as for example 1-butene, isobutene, 1-pentene and 1-hexene) instead of the corresponding alcohols, as described in patents U.S. Pat. No. 7,153,996 and WO 03/085071.

The processes carried out in two steps (synthesis of levulinic acid and subsequent esterification) also suffer from the same limitation in the yield of the one-step processes described above, again due to the acid-catalyzed decomposition reactions of the product. The limiting step is the transformation of the cellulose into levulinic acid for which yields of up to 74% are indicated (U.S. Pat. No. 4,897,497). Considering that for the subsequent esterification step yields of up to 85% are indicated (WO 2005/070867), the maximum final yield of the overall process is equal to 63%.

In the present invention, a process is described for the direct transformation of biomasses containing one or more polysaccharides, preferably biomasses containing a cellulose component, into esters of levulinic acid with high yields, using particular organic acid catalysts capable of minimizing the degradation reactions of the products.

The reaction is carried out in a single step using an alcohol as solvent and reagent, preferably selected from those obtained by fermentation from renewable sources. The esters of levulinic acid are thus produced using solely renewable raw materials.

With the process of the present invention, it is consequently now possible to obtain esters of levulinic acid in a single step with much higher yields than those obtainable with the known processes.

An object of the present invention therefore relates to a process for the direct synthesis of esters of levulinic acid from biomasses containing one or more polysaccharides which comprises putting the biomass in contact with an alcohol in the presence of an organic acid catalyst containing at least 10 carbon atoms.

The alcohol used has the formula R—OH, wherein R can be a linear, branched or cyclic aliphatic chain, preferably containing from 1 to 8 carbon atoms. According to a particularly preferred aspect, the alcohol contains from 2 to 4 carbon atoms and is preferably selected from ethanol, isopropanol or n-butanol.

The organic acid used as catalyst can belong to the group of alkyl- or arylsulfonic acids. Acids which can be conveniently used are 1-naphthalenesulfonic acid, 2-naphthalenesulfonic acid and 1,5-naphthalenedisulfonic acid. The preferred catalyst is 2-naphthalenesulfonic acid.

The concentration of the catalyst can range from 0.1 to 5% by weight with respect to the weight of the alcohol solution consisting of the alcohol and organic acid, preferably from 0.5 to 2% by weight.

Biomasses in which at least one polysaccharide contained in said biomass comprises hexose saccharide units are preferably used, and a even more preferred aspect is that at least one of said polysaccharides comprising hexose saccharide units is cellulose, hemicellulose, starch or mixtures thereof. In particular a particular preferred aspect is to use lignin-cellulose biomasses. As already mentioned, lignin-cellulose biomasses are characterized in that they contain three main components: cellulose, hemicellulose and lignin. Cellulose is the largest constituent of lignin-cellulose biomasses (30-60% by weight) and consists of linear glucose chains bound through bonds of the 1-4 β-glucoside type. Hemicellulose forms 10-40% of lignin-cellulose biomasses, it appears as a mixed polymer consisting of sugars with six carbon atoms and sugars with five carbon atoms.

Examples of lignin-cellulose biomasses which can be conveniently used in the process of the present invention can be:
products of crops expressly cultivated for energy use, including waste products, residues and scraps of said crops or their processing;
products of agricultural and forest cultivations, comprising wood, plants, residues and waste products of agricultural processing and forestry;
waste of agro-food products destined for human nutrition or zootechnics;
residues, not treated chemically, of the paper industry;
waste products coming from the differentiated collection of solid urban waste (e.g. urban waste of a vegetable origin, paper).

Crops for energy use which can be conveniently used can be, for example: miscanthus, foxtail millet and common cane.

Under the acid process conditions, there is the breakage of the polymeric chains of the sugars, the glucose is transformed into levulinic acid, by means of acid-catalyzed dehydration to give hydroxymethylfurfural and subsequent rearrangement with the formation of levulinic acid, which is esterified by the alcohol present in the reaction environment.

The alcoholysis process of the present invention is carried out in an autoclave by suspending the biomass in a solution containing the alcohol, used as solvent and reagent for the esterification of the levulinic acid, and the organic acid catalyst soluble in the alcohol.

The concentration of the biomass in the reaction mixture can vary from 5 to 30% by weight, preferably from 10 to 20% by weight with respect to the total weight of the reaction mixture.

The biomasses used as raw material of the hydrolysis process are preferably subjected to a preliminary grinding process to obtain particles having a diameter lower than 1 mm.

The reaction is carried out in an autoclave maintaining the reaction mixture under stirring at a temperature ranging from 160 to 230° C., preferably from 180 to 210° C.

The reaction time can range from 1 to 8 hours, preferably from 2 to 5 hours.

At the end of the transformation, the solid phase, consisting of lignin, is separated by filtration from the liquid phase, containing the ester of levulinic acid, the acid catalyst and by-products deriving from formic acid and furfural.

The liquid phase is evaporated, preferably at reduced pressure, by distilling the alcohol solvent until a biphasic system is formed, consisting of an alcohol phase, containing the concentrated acid catalyst (30-40% by weight), and a second organic phase comprising the ester of levulinic acid.

The phase containing the ester of levulinic acid is then separated and subjected to a subsequent purification step for the recovery of the residual alcohol and by-products. The purification can be conveniently effected, for example, by distillation.

The alcohol phase containing the catalyst can be reused directly in the reaction step, thus allowing the same catalyst to be recycled, in this way avoiding both consumption of the acid catalyst and the neutralization step of the products which, in the processes of the prior art, is generally effected with the addition of calcium hydroxide which consequently causes the coproduction of salts (CaSO₄).

The esters of levulinic acid thus obtained can be applied in various fields and in particular they can be well used as oxygenated components in the formulation of fuels for motor vehicles, with the aim of reducing particulate emissions. They can be particularly used as oxygenated components for both high-quality diesel fuel and in alkylated mixtures for air-engines.

### EXAMPLE 1

10 g of ground coniferous wood (particle dimension <1 mm) are added to a solution of 2 g of 2-naphthalenesulfonic acid in 100 ml of ethanol. The mixture is kept under stirring in an autoclave at 200° C. for 4 hours.

After cooling, the solid phase is separated by filtration and dried, obtaining 2.5 g of lignin with a purity >95%, corresponding to a yield to lignin >95% (the lignin content of the starting biomass is equal to 25% by weight).

The solution in ethanol is evaporated at reduced pressure until a biphasic system is formed, consisting of an alcohol phase (5 ml) containing 2-naphthalenesulfonic acid and a second organic phase (6 ml) containing ethyl levulinate.

After the separation, the phase containing the acid catalyst can be recycled in a subsequent alcoholysis cycle. The phase containing the ester of levulinic acid is distilled at reduced pressure obtaining ethanol and 4.3 g of ethyl levulinate (corresponding to a yield of 97% calculated with respect to the cellulose component of the starting biomass, which is equal to 50% by weight).

### EXAMPLE 2

10 g of ground coniferous wood (particle dimension <1 mm) are added to a solution of 2 g of 1,5-naphthalenedisulfonic acid in 100 ml of ethanol. The mixture is kept under stirring in an autoclave at 200° C. for 4 hours.

After cooling, the solid phase is separated by filtration and dried, obtaining 2.8 g of lignin with a purity equal to 89%, corresponding to a yield to lignin >95% (the lignin content of the starting biomass is equal to 25% by weight).

The solution in ethanol is evaporated at reduced pressure until a biphasic system is formed, consisting of an alcohol phase (5 ml) containing 1,5-naphthalenedisulfonic acid and a second organic phase (6 ml) containing ethyl levulinate.

After the separation, the phase containing the acid catalyst can be recycled in a subsequent alcoholysis cycle. The phase containing the ester of levulinic acid is distilled at reduced pressure obtaining ethanol and 4.1 g of ethyl levulinate (corresponding to a yield of 92% calculated with respect to the cellulose component of the starting biomass, which is equal to 50% by weight).

### EXAMPLE 3

10 g of ground coniferous wood (particle dimension <1 mm) are added to a solution of 2 g of 2-naphthalenesulfonic acid in 100 ml of 1-butanol. The mixture is kept under stirring in an autoclave at 200° C. for 4 hours.

After cooling, the solid phase is separated by filtration and dried, obtaining 2.7 g of lignin with a purity 92%, corresponding to a yield to lignin >95% (the lignin content of the starting biomass is equal to 25% by weight).

The solution in 1-butanol is evaporated at reduced pressure until a biphasic system is formed, consisting of an alcohol phase (5 ml) containing 2-naphthalenesulfonic acid and a second organic phase (6 ml) containing butyl levulinate.

After the separation, the phase containing the acid catalyst can be recycled in a subsequent alcoholysis cycle. The phase containing the ester of levulinic acid is distilled at reduced pressure obtaining 1-butanol and 4.5 g of butyl levulinate (corresponding to a yield of 85% calculated with respect to the cellulose component of the starting biomass, which is equal to 50% by weight).

### EXAMPLE 4

10 g of ground coniferous wood (particle dimension <1 mm) are added to a solution of 2 g of 2-naphthalenesulfonic acid in 100 ml of 2-propanol. The mixture is kept under stirring in an autoclave at 200° C. for 4 hours.

After cooling, the solid phase is separated by filtration and dried, obtaining 2.7 g of lignin with a purity 92%, corresponding to a yield to lignin >95% (the lignin content of the starting biomass is equal to 25% by weight).

The solution in 2-propanol is evaporated at reduced pressure until a biphasic system is formed, consisting of an alcohol phase (5 ml) containing 2-naphthalenesulfonic acid and a second organic phase (6 ml) containing isopropyl levulinate.

After the separation, the phase containing the acid catalyst can be recycled in a subsequent alcoholysis cycle. The phase containing the ester of levulinic acid is distilled at reduced pressure obtaining 2-propanol and 4.6 g of isopropyl levulinate (corresponding to a yield of 89% calculated with respect to the cellulose component of the starting biomass, which is equal to 50% by weight).

1) A process for the direct synthesis of esters of levulinic acid from biomasses containing one or more polysaccharides which comprises putting the biomass in contact with an alcohol in the presence of an organic acid catalyst containing at least 10 carbon atoms.

2) The process according to claim 1, wherein at least one polysaccharide contained in the biomass comprises hexose saccharide units.

3) The process according to claim 2, wherein at least one of said polysaccharides comprising hexose saccharide units is selected from cellulose, hemicellulose, starch or mixtures thereof.

4) The process according to one or more of the previous claims, wherein the biomass is a lignin-cellulose biomass.

5) The process according to claim 4, wherein the lignin-cellulose biomass is selected from:
products of crops expressly cultivated for energy use, including waste products, residues and scraps of said crops or their processing;
products of agricultural and forest cultivations, comprising wood, plants, residues and waste products of agricultural processing and forestry;
waste of agro-food products destined for human nutrition or zootechnics;
residues, not treated chemically, of the paper industry;
waste products coming from the differentiated collection of solid urban waste (e.g. urban waste of a vegetable origin, paper).

6) The process according to claim 5, wherein the crops expressly cultivated for energy use are miscanthus, fox-tail millet or common cane.

7) The process according to one or more of the previous claims, wherein the alcohol has the formula R—OH wherein R is a linear, branched or cyclic aliphatic chain.

8) The process according to claim 7, wherein R contains from 1 to 8 carbon atoms.

9) The process according to claim 8, wherein R contains from 2 to 4 carbon atoms.

10) The process according to claim 9, wherein R is ethyl, isopropyl, n-butyl.

11) The process according to one or more of the previous claims, wherein the organic acid is an alkylsulfonic or arylsulfonic acid.

12) The process according to claim 11, wherein the acid is selected from 1-naphthalenesulfonic acid, 2-naphthalenesulfonic acid and 1,5-naphthalenedisulfonic acid.

13) The process according to claim 12, wherein the acid is 2-naphthalenesulfonic acid.

14) The process according to claim 1, wherein the organic acid catalyst is used in a concentration ranging from 0.1 to 5% by weight with respect to the weight of the alcohol solution consisting of the alcohol and organic acid.

15) The process according to claim 14, wherein the concentration of the acid ranges from 0.5 to 2% by weight with respect to the weight of the alcohol solution consisting of the alcohol and organic acid.

16) The process according to claim 1, wherein the concentration of the biomass ranges from 5 to 30% by weight with respect to the total weight of the reaction mixture.

17) The process according to claim 16, wherein the concentration of the biomass ranges from 10 to 20% by weight.

18) The process according to one or more of the previous claims, carried out at a temperature ranging from 160 to 230° C.

19) The process according to claim 18, wherein the temperature ranges from 180 to 210° C.

20) The process according to claim 1 or 18, wherein the reaction time ranges from 1 to 8 hours.

21) The process according to one or more of the previous claims, wherein the resulting product is subjected to separation to remove the solid lignin phase, then evaporated until a biphasic system is formed, consisting of an alcohol phase containing the concentrated acid catalyst, which is recycled, and a second organic phase comprising the ester of levulinic acid.

22) A process for preparing oxygenated components for formulations of fuels for motor vehicles comprising the process according to one or more of the previous claims.
